(19) United States
(12) Patent Application Publication
(10) Pub. No.: US 2025/0111918 A1
(43) Pub. Date: Apr. 3, 2025

(54) SYSTEMS AND METHODS FOR OBTAINING DATA RELEVANT TO DIABETES MANAGEMENT USING LARGE LANGUAGE MODELS

(71) Applicant: Tandem Diabetes Care, Inc., San Diego, CA (US)

(72) Inventors: Josh JUSTER, San Diego, CA (US); Nathan PABRAI, Miami, FL (US)

(73) Assignee: Tandem Diabetes Care, Inc., San Diego, CA (US)

(21) Appl. No.: 18/477,369

(22) Filed: Sep. 28, 2023

(51) Int. Cl. G16H 20/17; G16H 20/60; G16H 40/63

(52) U.S. Cl. CPC G16H 20/17; G16H 20/60; G16H 40/63

(57) ABSTRACT

Systems and methods are provided including insulin pump systems for determining certain information about a user of a wearable insulin pump based on naturally spoken user data by applying the user data to a large language model (LLM) or other machine learning model to parse the user data into categories. For example, certain rules and/or constraints may be provided to the LLM and may cause the LLM or machine learning model to parse the user data into defined categories that may be used for updating a user record. Categories may include food consumption, activities, sleep, and/or pump operation information, for example. Additional data may be determined from the parsed data such as caloric information, exercise duration, sleep information and the like. The parsed categorized data and/or additional data may be used to adjust operation of the insulin delivery pump.

### TECHNICAL FIELD

This technology generally relates to the field of diabetes management. For example, systems and methods are provided herein for obtaining data relevant to diabetes management using a large language model.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a needle prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in monitoring technology, continuous glucose monitoring using a wearable patch including a small needle now provides on demand glucose monitoring for adjusting pump operation based on the glucose measurements. Additionally, pump operation is often adjusted based on information about the user including food consumption and activity such as exercise. Other information about the user such as sleep activity and/or information about the pump may be used to adjust pump operation to improve blood glucose levels of the user and/or health generally. Such information may be tracked manually by populating the information into user records and similar documents.

While users of insulin pumps may manually enter user data relevant to insulin pump operation into user records for determining adjusted pump operation based on this information, manually entering such information is time consuming and meticulous. Entering such information via voice transcription and even automated analysis systems has done little alleviate such difficulties due to limitations with traditional text recognition algorithms and keyword categorization systems.

Accordingly, there is a need for improved systems and methods for efficiently and accurately collecting and categorizing user data relevant to operation of an insulin delivery pump.

### SUMMARY

Provided herein are systems and methods for determining certain user information regarding a user of a wearable insulin pump based on naturally spoken by the user including applying the user data to a large language model (LLM) or other machine learning model to parse the user data into categories. Certain rules and/or constraints may be provided to the LLM and may cause the LLM or machine learning model to parse the user data into defined categories that may be used for updating a user record. Categories may include food consumption, activities, sleep, and/or pump operation information, for example. Additional data may be determined from the parsed data such as caloric information, exercise duration, sleep information and the like. The parsed categorized data and/or additional data may be used to adjust operation of the insulin delivery pump and otherwise improve health of the user.

A method for collecting user information is provided herein for determining parameters for adjusting operation of a user's insulin delivery pump. The method may include determining a large language model trained to process spoken data and associate categories to the spoken data, determining a set of rules adapted to cause the large language model to associate first portions of the spoken data with a first category corresponding to food and second portions of the spoken data with a second category corresponding to activity, determining user spoken data corresponding to the user of the insulin delivery pump, causing the large language model to process the user spoken data based on the set of rules, receiving food data associated with the first category and activity data associated with the second category, the food data and the activity data determined using the large language model and based on the user spoken data and the set of rules, and automatically generating insulin delivery parameters based on the food data and the activity data for adjusting operation of the insulin delivery pump.

The spoken data may be audio data and/or the method may further include transcribing the spoken data into text. The analyzed data may include food values. The food values may correspond to a calorie value and/or a carbohydrate value. The analyzed data may include a food type and/or the food values may be determined based on a database associating food types with food values. The method may further include sending the food data to a remote device and determining the food values from the remote device.

The insulin delivery parameters may be further based on the analyzed data. The method may further include determining activity values based on the activity data and/or the activity values may correspond to an amount of energy expended by the user. The method may further include causing a user device to present the insulin delivery parameters and/or a request to change operation of the insulin delivery pump based on the insulin delivery parameters. The method may further include automatically populating the user record corresponding to the user based on the food data and the activity data, and causing a user device to present at least a portion of the user record.

A system is further provided herein for collecting user information for determining parameters for adjusting operation of a user's insulin delivery pump. The system may include memory designed to store computer-executable instructions, and at least one computer processor designed to access memory and execute the computer-executable instructions to determine a large language model trained to process spoken data and associate categories to the spoken data, determine a set of rules adapted to cause the large language model to associate first portions of the spoken data with a first category corresponding to food and second portions of the spoken data with a second category corresponding to activity, determine user spoken data corresponding to the user of the insulin delivery pump, cause the large language model to process the user spoken data based on the set of rules, receive food data associated with the first category and activity data associated with the second category, the food data and the activity data determined using the large language model and based on the user spoken data and the set of rules, and automatically generate insulin delivery parameters based on the food data and the activity data for adjusting operation of the insulin delivery pump.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary insulin pump system for processing user data such as spoken data relevant to a user of a wearable insulin pump to determine categorized parsed data, in accordance with some aspects.

FIG. 2 illustrates an exemplary data flow depicting user data and rules processed by a large language model to determined categorized parsed data.

FIG. 3 illustrates an exemplary data flow for determining categorized parsed data relevant to a user of a wearable insulin pump in an exemplary insulin pump system.

FIG. 4 illustrates an exemplary process flow for determining categorized parsed data based on user data including spoken data using a large language model executed on a server.

FIG. 5 illustrates an exemplary process flow for determining categorized parsed data based on user data including spoken data using a large language model executed on a user device.

FIG. 6 illustrates a schematic block diagram of a server in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for collecting user data such as spoken data relevant to diabetes management and determining categorized parsed data based on the spoken data for populating a user record. For example, a user may speak into a device with a microphone and may generate audio data about meals and/or exercise. The audio data may be transcribed and processed by a large language model (LLM) which may also be provided certain rules and/or constraints for parsing the spoken data and determining categorized user data (e.g., food data, activity data, etc.) which may be populated into a user record used for diabetes management.

Other relevant information such as calorie or carbohydrate data or data about calories burned or exercise intensity may optionally be determined. The food data and/or activity data, as well as other relevant information determined (e.g., analyzed data) may be used to determine adjusted pump parameters (e.g., insulin delivery pump parameters and/or settings) for adjusting operation of the insulin delivery pump. The insulin pump system may seek permission from the user to adjust operation of the insulin delivery pump based on the adjusted pump parameters (e.g., insulin delivery parameters).

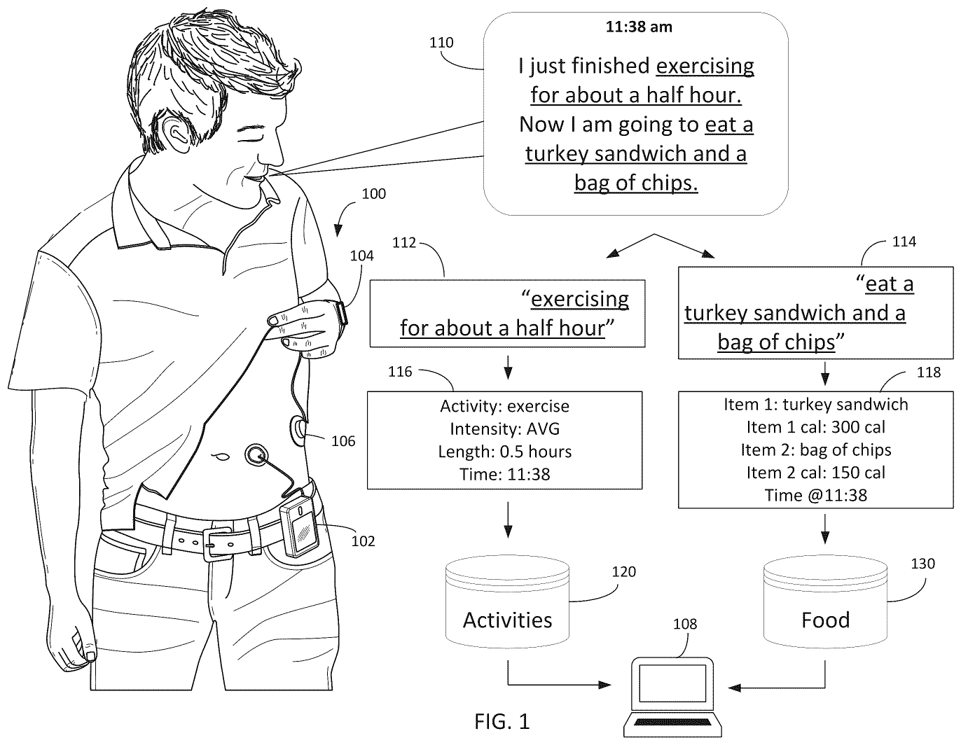

Referring now to FIG. 1, an insulin pump system for determining categories of parsed user data for updating a user record is depicted. Insulin pump system 100 may include insulin pump 102 as well as smart device 104. Insulin pump system 100 may also include or communicate with glucose monitor 106, a user device (e.g., smart phone) and/or remote device 108, which may be an insulin system server. Insulin pump system 100 may include greater or fewer devices than those illustrated in FIG. 1 and/or one or more device in FIG. 1 may be optional.

Insulin pump 102 may include a pump housing which may include a display and may house a pump designed to pump insulin into a user. For example, insulin pump 102 may be connected to a delivery component which may include tubing and/or a delivery patch having a needle or cannula for delivering insulin into a subcutaneous area of the user. Insulin pump 102 may include a compartment for holding a volume of insulin and may selectively deliver an amount of insulin (e.g., a bolus) to the user via the pump and delivery component 114.

Insulin pump 102 may, in one example, be the same as or similar to a t: slim X2™ insulin pump available from Tandem Diabetes Care, Inc. of San Diego, California and/or the insulin pump described in U.S. Patent App. Pub. No. 2014/0276423, published on Sep. 18, 2014 and assigned to Tandem Diabetes Care, Inc., and/or the insulin pump described in U.S. Patent App. Pub. No. 2011/014461, published on Jun. 16, 2011 and assigned to Tandem Diabetes Care, Inc., each of which are hereby incorporated by reference in their entirety. However, it is understood that insulin pump 102 may be any type of insulin pump for delivering insulin to the user and having the functionality described herein.

Insulin pump 102 may include one or more processors and memory as well as a communication chip for communicating with one or more periphery devices via a suitable wireless connection (e.g., Bluetooth, Bluetooth Low Energy (BLE), Wi-Fi Direct, any other near field communication protocol, and/or the like). Insulin pump 102 may optionally communicate directly with remote device 108, glucose monitoring sensor 106 and/or any other devices.

As shown in FIG. 1, insulin pump 102 may be an extracorporeal and may be worn by the user. The user may also have one or more periphery devices nearby. For example, the user may hold a mobile device in their pocket or personal bag (e.g., backpack or purse) and/or may wear smart device 104 (e.g., on their wrist).

The mobile device and/or smart device 104 may be any suitable smart device, smart phone, tablet, laptop, wearable, charging device, and/or smart sensor. The mobile device and/or smart device may include a computer processor, memory, microphone and/or a communication unit which may facilitate communication with pump 102, remote device 108, and/or any other devices (e.g., healthcare provider device, smart sensors such as glucose monitoring sensor 106, and/or any other devices).

The mobile device and/or smart device 104 may communicate with any devices in insulin pump system 100 and any other devices via any suitable wired or wireless system (e.g., Wi-Fi, cellular network, Bluetooth, Bluetooth Low Energy (BLE), near field communication protocol, etc.). Remote device 108 may be any computing device having a processor, memory and a communication unit. For example, remote device 106 may be one or more servers, datastores, or the like. Remote device 108 may communicate with any devices in insulin pump system 100 and any other devices via any well-known wired or wireless system (e.g., Wi-Fi, cellular network, Bluetooth, Bluetooth Low Energy (BLE), near field communication protocol, etc.).

To record user data (e.g., information) relevant to the user and their use of insulin delivery pump 102, a user may speak in natural spoken language regarding certain information about their activities, behaviors, experiences, and/or events throughout the day. For example, the user may speak into the microphone in smart device 104 and/or the mobile phone. The user may provide information about certain foods (e.g., food and/or drink) consumed, medication, exercise, sleep, operation of the insulin delivery pump, and the like. The audio data may optionally be transcribed. Alternatively, the user may type of such information (e.g., using the mobile device). Such data may then be sent to remote server 108 which may cause a LLM or other model to process the data.

As shown in FIG. 1, spoken data 110 may include “I just finished exercising for about a half hour” and “Now I am going to eat a turkey sandwich and a bag of chips.” Spoken data 110 may further include a time stamp (e.g., 11:38 am). Audio data of the spoken data may be captured and communicated to a server running a large language model (LLM) or alternative a model which may use machine learning (e.g., one or more neural networks) to parse user data (e.g., spoken data in audio or transcribed text form) and determine categories of text. For example, the LLM may be any suitable version of ChatGPT by OpenAI of San Francisco, California. It is understood, that the LLM or machine learning model may be any other LLM or machine learning model trained to process user data such as spoken data and parse such data into categories based on certain rules and/or constraints.

Spoken data 110 may be processed by the LLM or other machine learning model which may detect portions of the spoken data which may be associated with certain categories or labels based on known constraints or rules. As shown in FIG. 1, one category may be activities 120 and another category may be food 130. The LLM or other model may be instructed to detect and/or parse data that falls within the food category (e.g., any reference to food or drink, amount consumed, at what time, etc.). The LLM or other model may also be instructed to detect and/or parse data that falls within the activities category (e.g., exercise, intensity of exercise, length of exercise, calories burned, etc.). It is understood that different, additional, and/or other categories or labels may be used.

Using the LLM or other model, spoken data may be parsed into activity data 112 and food data 114. Food data 114 may include the text “exercising for about a half hour” and activity data 112 may include the text “eat a turkey sandwich and a bag of chips.” The LLM or other model may further be instructed to determine data 116 and data 118 which may be simplified or processed version of activity data for populating a user record and/or for further analysis. For example, data 116 may provide that activity data may be exercise data, intensity may be average, length may be 0.5 hours and time may be 11:38. The LLM or other model may include or may be in communication with a look up table, database, or other system for adding information about the exercise.

Additionally, data 118 may provide that one item eaten was a turkey sandwich and another item eaten was a bag of chips and that the time for both is 11:38. The LLM or other model may include or may be in communication with a look up table, database, or other system for adding information about the food consumed. For example, a database or lookup table may provide that a turkey sandwich is 300 calories and a bag of chips is 150 calories. Food data 114, activity data 114, data 116, and/or data 118 may be provided to server 108 for maintaining a record of such data (e.g., a user record). Such information may optionally be used to inform operation of the insulin delivery pump. Additionally, or alternatively, such information may be saved for retrospective analysis. For example, previously saved data may be compared to the newly obtained data for analysis and comparison.

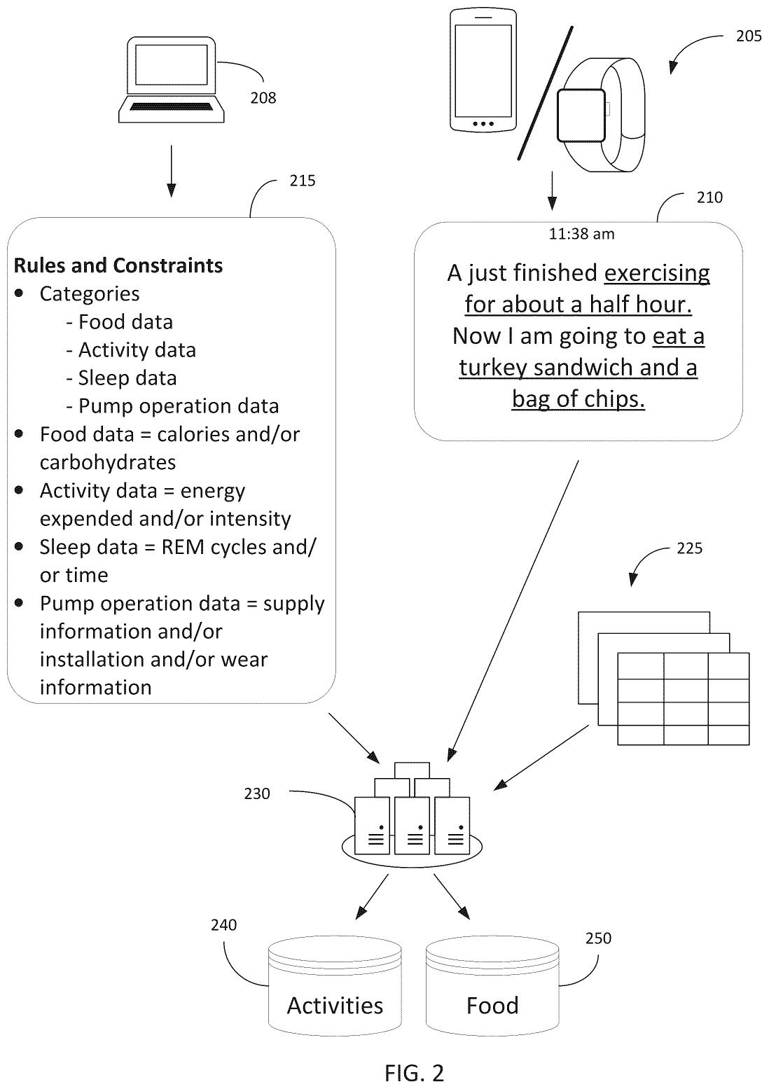

Referring now to FIG. 2, an exemplary data flow depicting user data and rules processed by a large language model to determine categorized parsed data is illustrated. As shown in FIG. 1, insulin system server 208, which may be any suitable or computing device having memory and a processor, may provide certain rules and/or constraints to a LLM server (e.g., server 230), which may process a LLM or machine learning model for parsing user data (e.g., spoken data) into categories based on rules and/or constraints from insulin system server 208.

Insulin system server 208 may send rules 215 to server 230 to define and/or set certain rules and/or constraints for server when processing user data using the LLM or model. For example, rules 215 may set forth certain categories (e.g., food data, activity data, sleep data, pump operation data). Rules 215 may further provide that food data (e.g., nutritional data) should include certain values such as fiber, fat, protein, sugar, calories and/or carbohydrates and the like, activity data should include certain values such as energy expended and/or intensity and the like, sleep data should include certain values about REM cycles and/or duration and the like, and/or pump operation data should include certain values such as supply information and/or installation and/or wear information and the like. Other categories may additionally or alternatively be used. For example, insulin data, medication data, liquid data, and device status data (e.g., changing a continuous glucose monitor (CGM) or infusion set) may be included.

Periphery devices 205 may be any user device used by the user (e.g., mobile device, smart device 104 of FIG. 1, or the like). Periphery devices may communicate user data 210, which may be spoken data such as audio data or transcribed data. Periphery device 205 may send user data 210 to insulin system server 208 which may forward the data to server 230. Server 230 may process user data 210 using the LLM or other model to parse user data 210 into categories based on rules 215. For example, portions of user data 210 may be parsed into activity data 240 and food data 250 based on rules 215. The LLM may be trained to determine certain user data may belong to a certain category based on the context or meaning of the information. For example, the user data may include the text “I lifted weights for an hour” and the LLM may be trained to determine that such information should be associated with a “Strength Training” category, which may be a predefined category

To provide additional data (e.g., analyzed data), system 225 may optimally be consulted. System 225 may be a remote device and/or may be a catalogue, lookup table, database or any other suitable repository of information. It is understood that system 225 may be incorporated in server 230 and/or may be a separate system that may be accessed to communicated with by server 230 and/or insulin system server 208. It is understood that different systems me be accessed by server 230, for different information (e.g., one system for food data and another system for activity data). In another example, system 225 may be smart device and/or a sensor device such as a glucose monitor, heart rate monitor, ECG monitor, or the like. Server 130 may then associate categories (e.g., activities 240 and/or food 250) with the parsed user data.

While FIG. 2 illustrates server 230 processing user data 210 and rules 215 using the LLM or other machine learning model, it is understood that the mobile device or smart device may alternatively process user data 210 and rules 215 using the LLM or other machine learning model. For example, periphery device 205 may be used to execute the LLM or other machine learning model.

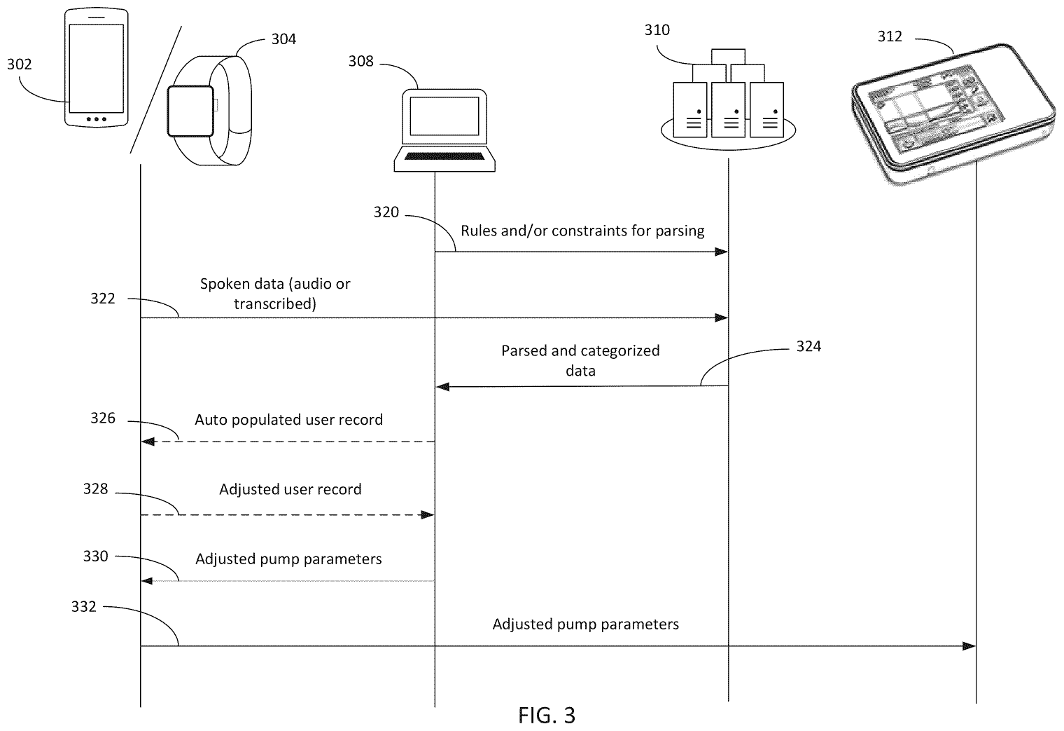

Referring now to FIG. 3, an exemplary data flow for determining categorized parsed data relevant to diabetes management in an exemplary insulin pump system is illustrated. To initiate the data flow of FIG. 3, insulin system server 308, which may be the same as insulin system server 208 of FIG. 1, may send rules and/or constraints to server 310, which may be the same as server 230 of FIG. 2. At block 322, mobile device 302 and/or smart device 304 may also send spoken data (e.g., audio data or transcribed text) to server 310.

At step 324, server 310 may send parsed and categorized data to insulin system server 308. For example, server 310 may send food data and/or activity data to insulin system server 308. Insulin system server 308 may automatically populate a user record with the parsed and/or categorized data and at optional step 326 may cause the user record to be presented on mobile device 302 and/or smart device 304. At optional step 328, mobile device 302 and/or smart device 304 may send information to adjust the user record (e.g., to fix any errors) to insulin system server 308.

At step 330, insulin delivery pump may determine adjusted pump parameters based on the parsed and/or categorized user data and send the adjusted pump parameters to mobile device 302 and/or smart device 304 and request approval to adjust the operation of the pump based on the adjusted pump parameters. At step 332, mobile device 302 and/or smart device 304 may send adjusted pump parameters to insulin delivery pump 312 for adjusting operation of the insulin delivery pump. Insulin delivery pump 312 may be the same as or similar to insulin delivery pump 102 of FIG. 1.

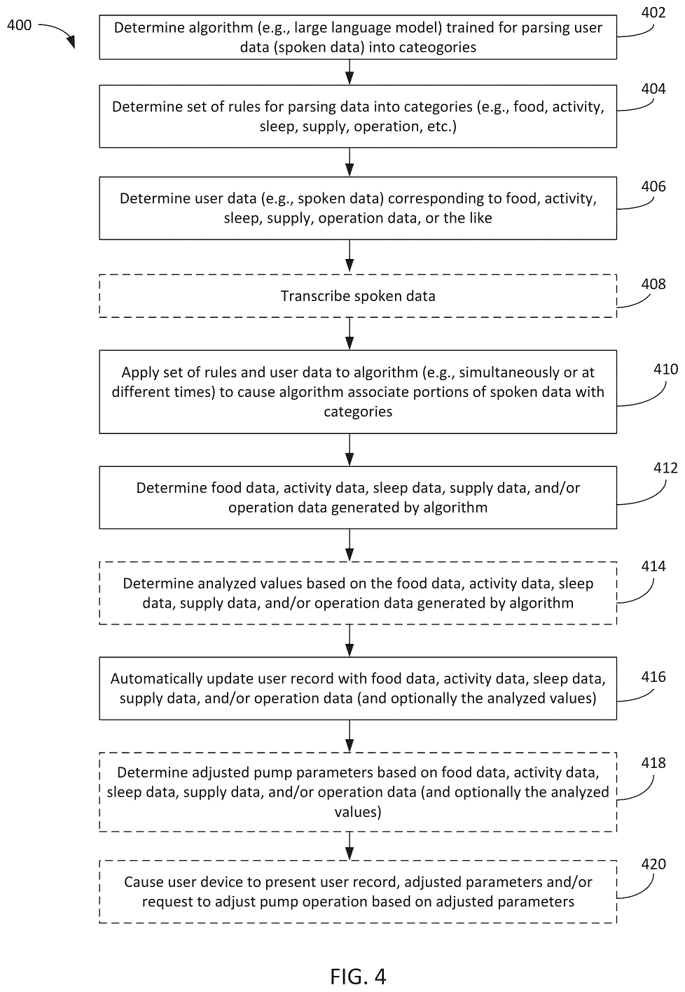

Referring now to FIG. 4, exemplary process flow for determining categorized parsed data based on user data including spoken data using a large language model is illustrated. Some or all of the blocks of process flow 400 in FIG. 4 may be performed in a distributed manner across any number of devices (e.g., insulin system server, mobile device, smart device, etc.). Some or all of the operations of process flow 400 may be optional and may be performed in a different order.

To initiate process flow 400, at block 402, computer-executable instructions stored on a memory of a device, such as an insulin delivery server, may be executed to determine an algorithm, such as a LLM or machine learning model, for parsing user data (e.g., spoken data) into categories. The spoken data may be audio data with natural language dialog from a user or may be transcribed audio. At block 404, computer-executable instructions stored on a memory of a device, such as an insulin delivery server, may be executed to determine a set of rules and/or constrains for parsing data into categories (e.g., food, activity, exercise, sleep, supply, pump operation, etc.) by the LLM or other machine learning model.

At block 406, computer-executable instructions stored on a memory of a device, such as an insulin delivery server, may be executed to determine user data (e.g., spoken data) corresponding to food intake, activity, user sleep, insulin pump supplies (e.g., insulin supply), operational data (e.g. pump clog occurrences), and the like). This information may be received from a smart device, for example, and may be audio data and/or transcribed text. At optional block 408, if the user data is audio data computer-executable instructions stored on a memory of a device, such as an insulin delivery server, may be executed to transcribe the audio data. It is understood that the user data may optionally be transcribed either by the insulin delivery server, by a third party system, and/or by a LLM.

At block 410, computer-executable instructions stored on a memory of a device, such as an insulin delivery server, may be executed to apply a set of rules and/or constraints and the user data to the algorithm. For example, the set of rules and/or constraints and/or the user data may be sent to a server or other computing device executing the LLM or machine learning model or may be sent at separate times. The insulin delivery server may then cause the LLM or machine learning model to process the user data based on the rules and/or constraints.

At block 412, computer-executable instructions stored on a memory of a device, such as an insulin delivery server, may be executed to determine parsed categorized data based on the user data and the rules and/or constraints (e.g., categorized food data, activity data, sleep data, supply data, and/or operation data generate). At block 414, computer-executable instructions stored on a memory of a device, such as an insulin delivery server, may be executed to determine analyzed values based on the parsed categorized user data. For example, the analyzed data may include additional information determined about the food activity and/or exercise (e.g., food values and/or exercise or activity values such as an amount of energy expended). In one example, a lookup table may be used to determine calories consumed based on a type of food or meal size. For example, the analyzed data may associate a food type with a food value (e.g., turkey sandwich is 300 calories).

At block 416, computer-executable instructions stored on a memory of a device, such as an insulin delivery server, may be executed to automatically update a user record with the parsed categorized data and/or analyzed data corresponding to the user that generated the user data. For example, process flow 400 may communicate with and/or may be incorporated into, in whole or in part, a diabetes management system such as or similar to the “Sugarmate” application by Tandem Diabetes Care, Inc. The Sugarmate application may integrate such information and may provide an activity feed, a nutritional database, and/or exercise information to the user, for example, and may record and analyze such information to find trends. The user record may be stored on the insulin system server. At optional block 418, computer-executable instructions stored on a memory of a device, such as an insulin delivery server, may be executed to determine adjusted pump parameters based on the parsed categorized used data (e.g., food data, activity data, sleep data, supply data, and/or operation data) and/or analyzed data.

At optional block 420, computer-executable instructions stored on a memory of a device, such as an insulin delivery server, may be executed to cause a user device such as a mobile device and/or smart device to present a user record or a portion of the user record, the adjusted parameters for operating the insulin delivery pump, and/or a request to adjust pump operation based on the adjusted parameters. The user may grant approval to adjust pump operation and/or adjust the user record.

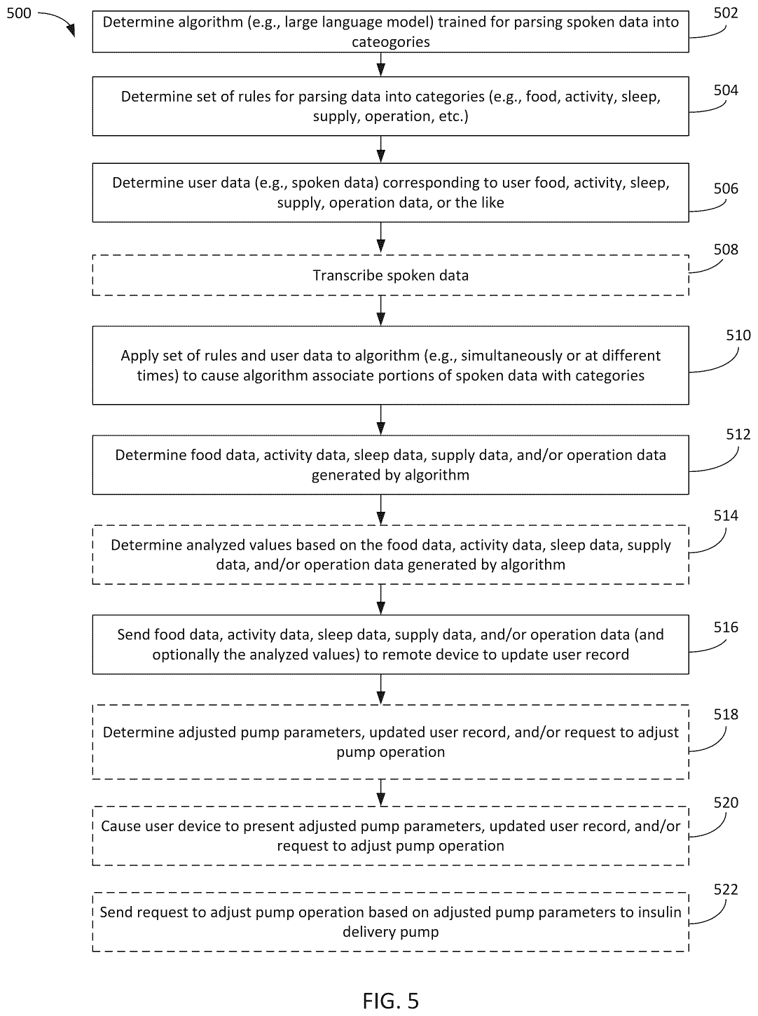

Referring now to FIG. 5, an exemplary process flow for the determining categorized parsed data based on user data including spoken data using a large language model executed on a user device (e.g., mobile device such as 302 of FIG. 3) is illustrated. Some or all of the blocks of process flow 500 in FIG. 5 may be performed in a distributed manner across any number of devices (e.g., insulin system server, mobile device, smart device, etc.). Some or all of the operations of process flow 500 may be optional and may be performed in a different order.

Process flow may be similar to process flow 400 of FIG. 4, but instead of a server executing the LLM or other machine learning model, a user device such as a mobile device may execute the LLM or other machine learning model. It is understood that the user data may be generated by the same device that executes the LLM and/or other machine learning model. Blocks 502-514 may be the same as blocks 402-412 of FIG. 4, but the user device may perform the steps instead of the insulin delivery server.

At block 516, computer-executable instructions stored on a memory of a device, such as a user device, may be executed to send parsed categorized user data and/or analyzed data to an insulin system server for updating the user record. At optional block 518, computer-executable instructions stored on a memory of a device, such as a user device, may be executed to determine adjusted pump parameters (e.g., based on the parsed categorized user data and/or analyzed data), an adjusted user record, and/or a request to adjust pump operation (e.g., from the insulin system server).

At optional block 520, computer-executable instructions stored on a memory of a device, such as a user device, may be executed to cause the user device to present adjusted pump parameters, the user record or a portion of the user record, and/or a request to adjust pump operation based on the adjusted pump parameters. At block 520, computer-executable instructions stored on a memory of a device, such as a user device, may be executed to cause the user device to send a request or instructions to adjust pump operation based on adjusted pump parameters to the insulin delivery pump.

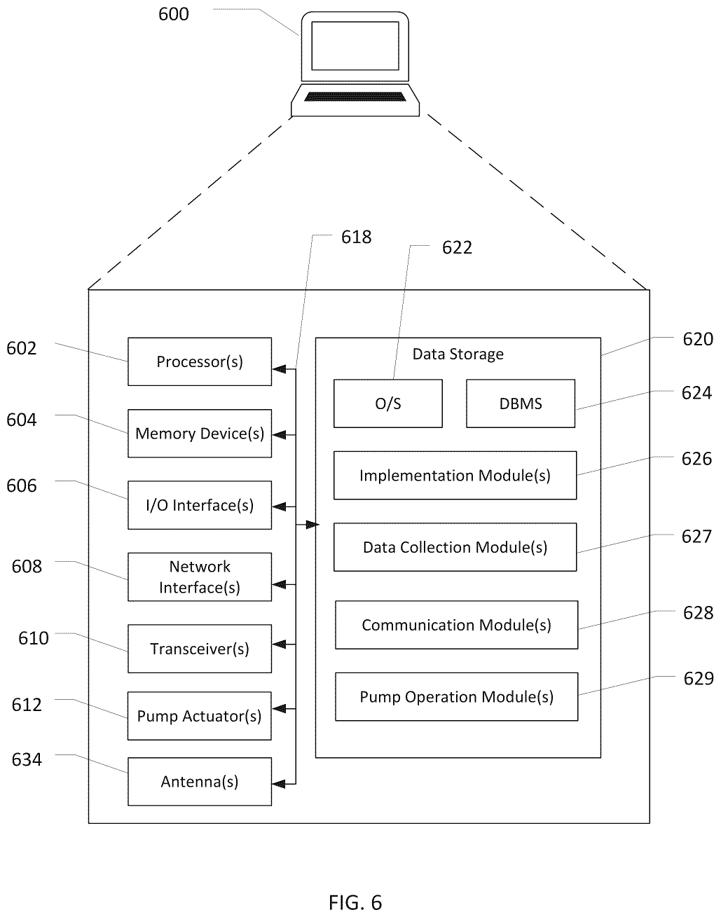

FIG. 6 is a schematic block diagram of illustrative insulin system server 600, which may be in communication with one or more periphery and/or remote devices, is illustrated. Insulin system server 600 may be the same or similar to insulin system server 108 of FIG. 1 or otherwise one or more of the insulin system servers of FIGS. 2-5. It is understood that an insulin system server 600 may alone or together with one or periphery or remote devices (e.g., mobile device) perform one or more of the operations of insulin system server 600. Alternatively, a mobile device (e.g., mobile device 302 of FIG. 3) may perform the operations and tasks of the insulin system server.

Insulin system server 600 may be designed to communicate with one or more periphery devices, remote devices, smart devices, computing devices, servers, other systems, or the like. Insulin system server 600 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, insulin system server 600 may include one or more processors 602, one or more memory devices 604 (also referred to herein as memory 604), one or more input/output (I/O) interface(s) 606, one or more network interface(s) 608, one or more transceiver(s) 610, one or more pump actuator(s) 612, one or more antenna(s) 634, and data storage 620. Insulin system server 600 may further include one or more bus(es) 618 that functionally couple various components of the insulin system server 600.

The bus(es) 618 may include at least one of a system bus, a memory bus, an address bus, or a message bus, and may permit exchange of information (e.g., data (including computer-executable code), signaling, etc.) between various components of the insulin system server 600. The bus(es) 618 may include, without limitation, a memory bus or a memory controller, a peripheral bus, an accelerated graphics port, and so forth. The bus(es) 618 may be associated with any suitable bus architecture including.

The memory 604 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein. The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

The data storage 620 may store one or more operating systems (O/S) 622; one or more optional database management systems (DBMS) 624; and one or more program module(s), applications, engines, computer-executable code, scripts, or the like such as, for example, one or more implementation modules 626, one or more data collection modules 627, one or more communication modules 628, and/or one or more pump operation modules 629. Some or all of these modules may be sub-modules. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

Referring now to other illustrative components depicted as being stored in data storage 620, O/S 622 may be loaded from data storage 620 into memory 604 and may provide an interface between other application software executing on insulin system server 600 and hardware resources of the insulin system server 600. More specifically, O/S 622 may include a set of computer-executable instructions for managing hardware resources of insulin system server 600 and for providing common services to other application programs (e.g., managing memory allocation among various application programs). In certain example embodiments, O/S 622 may control execution of the other program module(s) for content rendering. O/S 622 may include any operating system now known or which may be developed in the future including, but not limited to, any server operating system, any mainframe operating system, or any other proprietary or non-proprietary operating system.

Optional DBMS 624 may be loaded into the memory 604 and may support functionality for accessing, retrieving, storing, and/or manipulating data stored in memory 604 and/or data stored in data storage 620. DBMS 624 may use any of a variety of database models (e.g., relational model, object model, etc.) and may support any of a variety of query languages. DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 606 may facilitate the receipt of input information by insulin system server 600 from one or more I/O devices as well as the output of information from insulin system server 600 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into insulin system server 600 or may be separate.

Insulin system server 600 may further include one or more network interface(s) 608 via which insulin system server 600 may communicate with any of a variety of other systems, platforms, networks, devices, and so forth. Network interface(s) 608 may enable communication, for example, with one or more wireless routers, one or more host servers, one or more web servers, and the like via one or more of networks.

Antenna(s) 634 may include any suitable type of antenna depending, for example, on the communications protocols used to transmit or receive signals via antenna(s) 634. Non-limiting examples of suitable antennas may include directional antennas, non-directional antennas, dipole antennas, folded dipole antennas, patch antennas, multiple-input multiple-output (MIMO) antennas, or the like. Antenna(s) 634 may be communicatively coupled to one or more transceivers 610 or radio components to which or from which signals may be transmitted or received. Antenna(s) 634 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 610 may include any suitable radio component(s) for, in cooperation with the antenna(s) 634, transmitting or receiving radio frequency (RF) signals in the bandwidth and/or channels corresponding to the communications protocols utilized by insulin system server 600 to communicate with other devices. Transceiver(s) 610 may include hardware, software, and/or firmware for modulating, transmitting, or receiving-potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 610 may further include hardware, firmware, or software for receiving GNSS signals. Transceiver(s) 610 may include any known receiver and baseband suitable for communicating via the communications protocols utilized by the insulin system server 600. The transceiver(s) 610 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 626 may include computer-executable instructions, code, or the like that responsive to execution by one or more of the processor(s) 602 may perform functions including, but not limited to, overseeing coordination and interaction between one or more modules and computer executable instructions in data storage 620, determining user actions, determining actions associated with user interactions, determining actions associated with user input, initiating commands locally or at periphery and/or remote devices, and the like.

The data collection module(s) 627 may include computer-executable instructions, code, or the like that responsive to execution by one or more of the processor(s) 602 may perform functions including, but not limited to causing a LLM or machine learning model to parse user data into parsed categorized used data and may update a user record with the parsed categorized data and/or use such data to determine adjusted pump parameters for operating the insulin delivery pump.

Communication module(s) 628 may include computer-executable instructions, code, or the like that responsive to execution by one or more of the processor(s) 602 may perform functions including, but not limited to, communicating with one or more devices, for example, via wired or wireless communication, communicating with mobile devices, smart devices, remote devices, charging devices, computing devices, smart sensors and/or any other devices.

Pump operation module(s) 629 may include computer-executable instructions, code, or the like that responsive to execution by one or more of the processor(s) 602 may perform functions including, but not limited to, overseeing, monitoring, and/or operating the pump and pump actuators for select delivery of a volume of insulin based on pump parameters for the insulin delivery pump (e.g., pump settings).

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for collecting user information for determining parameters for adjusting operation of a user's insulin delivery pump, the method comprising:
determining a large language model trained to process spoken data and associate categories to the spoken data;
determining a set of rules adapted to cause the large language model to associate first portions of the spoken data with a first category corresponding to food and second portions of the spoken data with a second category corresponding to activity;
determining user spoken data corresponding to the user of the insulin delivery pump;
causing the large language model to process the user spoken data based on the set of rules;
receiving food data associated with the first category and activity data associated with the second category, the food data and the activity data determined using the large language model and based on the user spoken data and the set of rules; and
automatically generating insulin delivery parameters based on the food data and the activity data for adjusting operation of the insulin delivery pump.

2. The method of claim 1, wherein the spoken data is audio data, the method further comprising transcribing the spoken data into text.

3. The method of claim 1, further comprising determining analyzed data based on the food data, wherein the analyzed data comprises food values.

4. The method of claim 3, wherein the food values correspond to a calorie value and/or a carbohydrate value.

5. The method of claim 4, wherein the analyzed data comprises a food type and wherein the food values are determined based on a database associating food types with food values.

6. The method of claim 3, further comprising:
sending the food data to a remote device; and
determining the food values from the remote device.

7. The method of claim 3, wherein the insulin delivery parameters are further based on the analyzed data.

8. The method of claim 1, further comprising determining activity values based on the activity data, the activity values corresponding to an amount of energy expended by the user.

9. The method of claim 1, further comprising causing a user device to present the insulin delivery parameters and/or a request to change operation of the insulin delivery pump based on the insulin delivery parameters.

10. The method of claim 1, further comprising:
automatically populating a user record corresponding to the user based on the food data and the activity data; and
causing a user device to present at least a portion of the user record.

11. A system for collecting user information for determining parameters for adjusting operation of a user's insulin delivery pump, the system comprising:
memory configured to store computer-executable instructions; and
at least one computer processor configured to access memory and execute the computer-executable instructions to:
determine a large language model trained to process spoken data and associate categories to the spoken data;
determine a set of rules adapted to cause the large language model to associate first portions of the spoken data with a first category corresponding to food and second portions of the spoken data with a second category corresponding to activity;
determine user spoken data corresponding to the user of the insulin delivery pump;
cause the large language model to process the user spoken data based on the set of rules;
receive food data associated with the first category and activity data associated with the second category, the food data and the activity data determined using the large language model and based on the user spoken data and the set of rules; and
automatically generate insulin delivery parameters based on the food data and the activity data for adjusting operation of the insulin delivery pump.

12. The system of claim 11, wherein the spoken data is audio data and wherein the at least one computer processor is further configured to access memory and execute the computer executable instructions to transcribe the spoken data into text.

13. The system of claim 11, wherein the at least one computer processor is further configured to access memory and execute the computer executable instructions to determine analyzed data based on the food data, wherein the analyzed data comprises food values.

14. The system of claim 13, wherein the food values correspond to a calorie value and/or a carbohydrate value.

15. The system of claim 14, wherein the analyzed data comprises a food type and wherein the food values are determined based on a database associating food types with food values.

16. The system of claim 13, wherein the at least one computer processor is further configured to access memory and execute the computer executable instructions to:
send the food data to a remote device; and
determine the food values from the remote device.

17. The system of claim 13, wherein the insulin delivery parameters are further based on the analyzed data.

18. The system of claim 11, wherein the at least one computer processor is further configured to access memory and execute the computer executable instructions to determine activity values based on the activity data, the activity values corresponding to an amount of energy expended by the user.

19. The system of claim 11, wherein the at least one computer processor is further configured to access memory and execute the computer executable instructions to cause a user device to present the insulin delivery parameters and/or a request to change operation of the insulin delivery pump based on the insulin delivery parameters.

20. The system of claim 11, wherein the at least one computer processor is further configured to access memory and execute the computer executable instructions to:
automatically populate a user record corresponding to the user based on the food data and the activity data; and
cause a user device to present at least a portion of the user record.
